(19) United States
(12) Patent Application Publication
(10) Pub. No.: US 2006/0141571 A1
(43) Pub. Date: Jun. 29, 2006

(54) Method for promoting cell growth and increasing the production of the expressed target gene products

(75) Inventors: Yun-Peng Chao, Taichung City (TW); Zei Wen Wang, Tainan City (TW); Yuhsin Chen, Changhua County (TW)

Correspondence Address: BACON & THOMAS, PLLC, 625 SLATERS LANE, FOURTH FLOOR, ALEXANDRIA, VA, 22314, US

(73) Assignee: Taichun District Agricultural Research & Extension Station, Council of Agriculture, Executive Yuan, Changhua County (TW)

(21) Appl. No.: 11/286,419

(22) Filed: Nov. 25, 2005

(30) Foreign Application Priority Data
Dec. 23, 2004 (TW) 93140200

(51) Int. Cl. C12P 21/06; C12N 9/24; C12N 5/06; C12N 5/08; C12N 5/04; C12N 1/18

(52) U.S. Cl. 435/69.1; 435/200; 435/252.33; 435/254.2; 435/366; 435/348; 435/419

(57) ABSTRACT

The present invention is related to a method to promote cell growth, comprising: (a) cloning one or more aspartase coding sequence into a vector to become a recombinant vector, wherein the recombinant vector including a promoter and one or more aspartase coding sequences manipulated to link and insert into the downstream of said promoter; (b) transforming the recombinant vector into host cells and producing recombinant host cells; and (c) culturing the host cells in culture medium and making the cells to express aspartase, then promoting said recombinant host cells growth. The aspartase can not only promote cell growth but also increase the amount of the target gene expression.

### BACKGROUND OF THE INVENTION

1. Field of the Invention

The present invention is directed to the construction of recombinant vectors containing aspartase coding sequence and methods of utilizing said recombinant vectors to produce recombinant cells, thereby promoting the cell growth and increasing the amount of the target gene expression in the cells.

2. Description of Prior Art

Recombinant DNA technology has been applied to producing a large amount of recombinant polypeptide/protein in the cells. Its basic principle is to clone target genes which include industrial and agricultural enzymes, therapeutic proteins, antigenic polypeptides, antibodies, and so on into a suitable vector. Accordingly, recombinant vectors obtained from said process are transformed into competent host cells. Nevertheless, many factors lead to plasmid instability and the loss of recombinant vectors from transformed host cells as a consequence. Direct insertion of target gene into chromosome of cells can be used to solve this problem. The method of gene insertion can be achieved by phage/virus infection, transposition by transposons, or homologous recombination. (Haldimann et al. (2001), J. Bacteriol., 183: 6384-6393) As a result, recombinant host cells formed through above process can be cultured under suitable culture mediums and culture conditions, and be induced to express the product of target genes in a massive amount.

Among the host cells used for producing recombinant peptide/protein, Escherichia coli is the most widely used and the most successful producer cell. There are large numbers of vectors developed from E. coli, mainly including high-copy-number (ex. ColE1), middle-copy-number (ex. P15A) and low-copy-number plasmid (ex. pSC101) (Makrides et al. (1996), Microbiol. Rev., 60:512-538). These vectors are usually constructed to carry an inducible artificial promoter, including the most frequently ones, for example, lac-lpp-trp-tac-trc-araBAD-λP_{R}P_{L}-T7 A1, and T7 promoter. The way to induce these promoters can be achieved by adding chemicals like isopropyl-β-D-thiogalactopyranoside (IPTG), lactose, and arabinose or by changing the temperature (Makrides et al. (1996), Microbiol. Rev., 60: 512-538). Accordingly, the target gene cloned in the downstream of the artificial promoter can fulfill the purpose of regulating the expression of the target gene by timely switching on the induction mechanism according to the regulatory characteristics of the promoter.

Generally speaking, almost all genes from different organisms can be expressed in E. coli, and related manipulating techniques have been well developed nowadays. However, there are still some problems to be solved, for example, when a large amount of recombinant peptide/protein is induced, host cells are often and severely afflicted by metabolic burden, namely stress responses triggered in host cells, and thereby inhibiting cell growth (Schweder et al. (2002), Appl. Microbiol. Biotechnol., 58:330-337). The phenomenon frequently induces the production of large numbers of heat shock proteins in cells. As a result, recombinant peptide/protein will be attacked and decomposed by protease (Bahl et al. (1987), Gene Dev., 1:57-64), and cell growth is impaired (Kurland et al. (1996), Mol. Microbiol., 21:1-4). On the other hand, the phenomenon also causes the destroy of rRNAs and the disruption of ribosome, which leads to cell lysis (Dong et al. (1995), J. Bacteriol., 177:1497-1504). It is worthy to mention that it is highly possible to induce stress response within producer cells upon induction for protein overproduction, no matter that the peptide/protein is toxic or related to the host cells' metabolism. As a consequence, it makes a large production of recombinant peptide/protein hardly achievable.

Besides, a large amount of recombinant peptide/protein expressed in E. coli usually results in the formation of inclusion body. Previous researches show that a large production of thioredoxin in E. coli can improve the reduced state of cells, which is helpful for the production of soluble proteins of eukaryotic cells (LaVallie et al. (1993), Bio/Technology, 11:187-193 ;Yasukawa et al. (1995), J. Biol. Chem., 270:25328-25331). In normal cell physiological condition, the reaction cycle involved by glutathione and glutaredoxine can produce two intracellular enzymes with the formation of disulfide bonds in E. coli. i.e. ribonucleotide reductase and oxidative response transcription factor (Aberg et al. (1989), J. Biol. Chem., 264:12249-12252; Zheng et al. (1998), Science, 279:1718-1721).

In industrial bioprocess, the issue of oxygen supply for the aerobic fermentation of recombinant cells could largely affect cell growth and the amount of recombinant peptide/protein production. Dissolved oxygen concentration of fermentation broth can be improved by mechanical method such as increasing stirring velocity increasing aeration volume or the proper design of stirring blades. However, the effect of these methods is limited in the case of the operation of large-scale fermenters, and it may increase the fermentation cost because of energy waste. Especially in high-cell-density fermentation, high oxygen consumption by cells limits dissolved oxygen concentration of fermentation broth and restricts cell growth and the production of recombinant peptide/protein. Khosla et al. found that the expressing of the hemoglobin gene from Vitreoscilla sp. in E. coli. can improve cell growth and the total cell mass under the hypoxic condition of fermentation (Khosla and Bailey (1988), Nature, 331:633-635).

Vitreoscilla sp. is filamentous aerobic bacteria which can grow in oxygen-poor environments. Under low dissolved oxygen condition, Vitreoscilla sp. can be induced to produce soluble hemoglobin which is proved homologous to eukaryotic hemoglobins by spectral, structural, and kinetic analysis (Webster et al. (1974), J. Biol. Chem. 249:4257-4260; Wakabayashi et al. (1986), Nature, 322:481-483; Orii et al. (1986), J. Biol. Chem. 261:2978-2986).

Khosla et al. have disclosed in U.S. Pat. No. 5,049,493 that the transformed host cell containing Vitreoscilla sp. hemoglobin sequence can promote host cell growth and increase the production of cell metabolite and proteins. According to another literature, E. coli producing Vitreoscilla sp. hemoglobin can effectively increase the amount of the cell's protein (Khosla et al. (1990), Bio/Technology, 8:849-853) and recombinant α-amylase (Khosravi et al. (1990), Plasmid, 24:190-194) under low dissolved oxygen condition. Moreover, when using Bacillus subtilis cells and Chinese hamster ovary cells as host cells, under oxygen-limited condition, recombinant cells containing Vitreoscilla sp. hemoglobin similarly produce larger amounts of recombinant proteins (Kallio and Bailey (1996), Biotechnol. Prog., 12: 31-39; Pendse and Bailey (1994), Biotehnol. Bioeng., 44:1367-1370)

In practical operation of fermentation process, the use of the fed-batch fermentation can effectively achieve high-cell-density culture, therefore increasing cell mass per fermentation volume and total protein production. However, the aerobic fermentation of cells often results in the production of wasted metabolites such as organic acid, in which acetic acid can destroy electron-transport chain system (or cellular respiratory system) and affect ATP production. According to the inhibitory mechanism of acetic acid to cell normal physiology (Baronofsky et al., (1984), Appl. Environ. Microbiol. 48 (6) :1134-1139; Luli and Strohl (1990), Appl. Environ. Microbiol. 56:1004-1011) disclosed in prior art, acetic acid exists in ionized (CH₃COO⁻) and proton type (CH₃COOH) at neutral pH. Acetic acid of proton type having weak hydrophobic nature can penetrate cell membrane to enter cell and dissociate to CH₃COO⁻and H⁺ intracellularly (pH 7.5) ; therefore, the intracellular pH value is lowered which diminishes Δ pH between inner side and outer side of cell membrane. This will lead to the reduced generation of proton motive force and decrease cell energy production.

Under aerobic conditions, the mechanism of forming intracellular acetic acid is still unknown. Nevertheless, it's generally said that the combined act of the glucose consumption rate out of a suitable control and the inefficient function of the tricarboxylic acid cycle (TCA cycle) will lead to unbalanced distribution of carbon flux between glycolysis and TCA cycle; consequently, acetic acid is produced intracellularly (EI-Mansi and Holms, (1989) J. Gen. Microbiol. 135:2875-2883). It is common to reduce acetic acid production disclosed in prior art by using mutant strains with low glucose uptake rate (Chou et al. (1994), Biotechnol. Bioeng. 44:952-960) or the use of fermentation strategies including DO stat, pH stat, and substrate feeding to control cell growth rate (Lee (1996), Trends Biotechnol. 14:98-105).

There are lots of factors affecting acetic acid production such as growth rate, culturing medium, temperature, dissolved oxygen, and cell itself, etc. However, the major pathway for acetic acid production is via the reversible reaction of phosphotransacetylase (pta) and acetate kinase (ack). Hence, prior researches reported the utilization of genetic techniques to construct pta or ack knock-out strains (Dedhia et al. (1994), Biotechnol. Bioeng. 44:132-139; Diaz-Ricci et al. (1991), Biotechnol. Bioeng. 38:1318-1324 ) or to convert acetic acid and its precursor into other less toxic substances (Aristidou et al. (1994), Biotechnol. Bioeng. 44:944-951; Aristidou et al. (1995), Biotechnol. Prog. 11:475-478) in order to reduce the accumulation of acetic acid in cell, and furthermore, to improve cell growth and recombinant protein production.

Moreover, prior arts claimed to improve the utilization efficiency of intracellular carbon resource for the purpose of curtailing carbon waste by manipulation of the anaplerotic pathway. E. coli makes use of so called “phosphotransferase (PTS) system” to intake glucose from the surroundings. Upon the transport of glucose across the cell membrane, glucose is phosphorylated to become glucose 6-phosphate (G6P) by the acceptance of phosphorous molecule from intermediate metabolite, phosphoenolpyruvate (PEP), via the conversion reaction of PTS system. Through a serious steps in glycolysis, oxidation of G6P gives PEP which is further converted to oxaloacetate (OAA) to supplement the carbon resource of TCA cycle by phosphoenolpyruvate carboxylase(ppc) (cf. FIG. 1). Chao et al. found that increasing activity of ppc can lead carbon flux into TCA cycle instead of acetic acid forming pathway and further deviate PEP from PTS system to lower glucose consumption rate. As a result, cell growth yield (i.e. cell mass produced per gram glucose consumed) doubles. (Chao and Liao (1993), Appl. Environ. Microbiol. 59:4261-4265). Similarly, the research of Farmer et al. also reported that the simultaneous increase in ppc and isocitrate lyase in TCA cycle can effectively control cellular acetic acid production (Farmer and Liao (1997), Appl. Environ. Microbiol. 63:3205-3210). On the other side, the work by March et al. demonstrated that the expressing of pyruvate carboxylase in E. coli can improve the utilization efficiency of glucose and reduce the production of acetic acid, resulting in the increase of 68% recombinant protein production (March et al. (2002) Appl. Environ. Microbiol. 68:5620-5624).

Due to the strong dependence of high productivity of recombinant peptide/protein on the competitiveness of bio-industry, it's evident that the subject of increasing the production of recombinant peptide/protein will become a study of focus. There is still a big gap for the improvement of cell growth and recombinant protein production. Therefore, it is valuable to explore new technologies to fulfill these goals.

### SUMMARY OF THE INVENTION

It is known that intracellular protein synthesis is a high-energy-requiring process and the generation of intracellular energy depends on the catabolism of carbon sources. To improve recombinant protein production in cells, applicants believe that, in one aspect, more carbon resources obtained by cells within a limited period of time is necessary; in another aspect, utilization of the acquired carbon resources to effectively generate more energy is needed.

Cell can obtain energy by the carbon-decomposed pathway. For cellular growth, glucose is a good carbon source due to more production of precursor metabolites and high energy production per unit carbon mass. Nevertheless, while using glucose, a mechanism of preventing the use of other carbon sources comes along (Postma et al. (1993) Microbiol. Rev. 57:534-594). For example, E. coli intakes glucose outside the cell via PTS system, yet, the activity of permease for transporting other carbon sources, such as lactose, melibiose, maltose, and glycerol, etc., is inhibited. The result will allow E. coli to have a preference for glucose, and this underlying mechanism is called “inducer exclusion”. In addition, the activity of adenylate cyclase is inhibited when PTS system in E. Coli is functioning. This will reduce the intracellular concentration of cyclic AMP (cAMP) and further suppress a lot of gene expression including permease genes for transporting carbon source, (like intermediate of TCA cycle, xylose, rhamnose, and galactose) thereby making cells fail to utilize carbon sources other than glucose; and this is called “catabolite repression”. In addition, as commonly recognized, the aerobic growth of E. coli on glucose will largely excrete acetate, and this is a manifestation of imbalanced flux between glycolysis and TCA cycle. Accordingly, the result may restrict the production of recombinant proteins in E. coli, due to the limited amounts of precursor metabolites produced in TCA cycle.

According to applicants' analysis (see FIG. 1), while using glucose as carbon source, if the cell has aspartase activity, extracellular aspartate can be transported into the cell and decomposed to produce fumarate, the intermediate of-TCA cycle, and the cell can get more carbon sources including glucose and fumarate at the same time. On the other hand, the formation of fumarate will be converted to OAA in several steps in TCA cycle pathway and nicotinamide-adenine dinucleotide (NADH) is produced to have the cell gain more energy. Moreover, an extra supply of intermediate metabolites in TCA cycle is achieved.

The present invention is intended to provide a method to improve cell growth, comprising: (a) cloning an aspartase nucleic acid sequence to the vector to form a recombinant vector; (b) transforming the recombinant vector from step (a) into a host cell to form a recombinant host cell; and (c) culture the recombinant host cell from step (b) in the medium to express the aspartase gene and improve the growth of said recombinant host cell.

The recombinant vector is characterized with a promoter, aspartase nucleic acid sequence code, and an origin of replication. Aforesaid nucleic acid sequence can be manipulated to connect and be inserted to the downstream of aforesaid promoter. The recombinant vector carries high copy number or low copy number of aspartase nucleic acid sequence depending on different types (such as, pMB1 type, pBR322 type, p15A type, pSC101 type, R1 type, RK2 type, R6K type, F type, or pSF1010 type) of origin of replication. The vectors contain those used in general genetic engineering, like bacteriophages, plasmids, cosmids, viruses, or retroviruses. Furthermore, the aspartase nucleic acid sequence used in the present invention is from E. coli, bacteria, yeasts, fungi, insects, plants, animals, and/or human cells, preferably from E. coli.

Aforesaid promoter of the recombinant vectors in the present invention intends to control gene expression of aspartase. The promoter is IPTG-inducible promoter, constitutive promoter or other controlling promoter, such as tac promoter, T7 promoter, T7 A1 promoter, lac promoter, trp promoter, trc promoter, araBAD promoter, or λP_{R}P_{L} promoter etc.

Aforesaid host cells of the method in the present invention include prokaryotic cells or eukaryotic cells. Prokaryotic cells suitable for, but is not limited to, the present invention are E. coli, Bacillus subtilis, Lactobacillus sp., Streptomyces sp., and Salmonella typhi; Cyanobacteria; Actinomycetes etc. Eukaryotic cells suitable for, but is not limited to, the present invention are Saccharomyces cerevisiae or Pichia pastoris; plant cells are derived from gynosperms or angiosperms, preferably monocots or dicots, especially crops; and the cells are taken from plants' roots, stems, leaves or meristem parts, and cultured as protoplasts, suspension cells or callus; insect cells are derived from S2 cells of fruit fly and Sf21 or Sf9 cells of Spodoptera frugiperda. Animal cells are cultured cells or in vivo cells including CHO, BHK, Hela etc.

Furthermore, the recombinant host cells are cultured in a suitable medium to allow the expression of the gene. Suitable media and conditions for recombinant host cells are well known in the skilled art, such as, culturing the host cells in the frequently used fermenting bioreactors, shake flasks, test tubes, micro-titer plates, flattened Petri dishes, and the culture is under suitable temperature, pH, and oxygen content for recombinant host cell growth. Medium used for culturing host cells contain carbon sources (such as glucose, lactose, sucrose, molasses, starch and cereal grains etc.), nitrogen sources (like, ammonium salts, urea, nitrates, corn steep liquor, soybean meal and yeast extract), phosphate, sulfate, growth factor (such as vitamin, amino acid, nucleic acid etc.), and trace amount of metal (like, potassium, magnesium, calcium, ferrum, zinc, sodium, cobalt, manganese, copper etc.) Shake flasks are used in the steps of the method in the present invention, and used medium can be any medium with aspartate, such as LB, LB with glucose, M9 with glucose and aspartate, or M9 with glucose, yeast extract with aspartate. The LB consists of yeast extract (5 g/L), tryptone (10 g/L), NaCl (5 g/L), thus it is said LB should contain aspartate. Moreover, said M9 comprises Na₂HPO₄ (6 g/L), KH₂PO₄ (3 g/L), NaCl (0.5 g/L), NH₄Cl (1 g/L), MgSO₄·7H₂O (1 mM), CaCl₂ (0.1 mM).

The other purpose of the present invention is to provide a method to increase the expression of target gene, and the method comprises steps as follows: (a) constructing a recombinant vector with aspartase nucleic acid sequence code; (b) constructing a recombinant vector with desired expressing target gene; (c) transforming the vectors from both steps (a) and (b) to a host cell and form a recombinant host cell; and (d) culturing the recombinant host cell from step (c) in the medium and inducing said recombinant cell to express aspartase and desired target gene. Aforesaid aspartase can increase the production of desired target gene in the recombinant host cell.

The construction of the vector, like recombinant vector of aforesaid method, has a promoter to regulate the gene expression of aspartase. The promoter is IPTG-inducible promoter, constitutive prompter or other controlling promoter. The aspartase nucleic acid sequence is from E. coli, bacteria, yeasts, fungi, insects, plants, animals, and/or human cells.

Aforesaid target gene products include recombinant proteins or peptides. The recombinant proteins are various type of proteins including proteins locating on cytoplasm or periplasm, proteins on cell membrane or extracellular proteins, and enzymes used for industry, agriculture, food, environment, fishery and graziery, especially medical proteins and peptides, like interferon, interleukin, animal or human hormone, immuno-antigen and antibody. Additionally, the desired recombinant proteins in the present invention contain heterologous proteins, like Aequorea green fluorescent protein, or homologous proteins, like β-galactosidase.

The method of co-transforming aforesaid plasmids into a host cell includes phosphocalcium or phosphochloride transfection, electroporation, microinjection, particle bombardment, liposome-mediated transfection, bacteriophage transfection, retrovirus or other virus transduction, protoplast fusion, Agrobacterium-mediated transformation or other methods. The host cells may be like the said bacteria, yeasts, fungi, plant cells, insect cells or mammalian cells. Aforesaid aspartase can increase the expression of desired target gene in the recombinant cells. Furthermore, shake flasks are used in the steps of the method in the present invention and the used medium can be any medium with aspartate such as, LB, LB with glucose, M9 with glucose and aspartate or M9 with glucose, yeast extract with aspartate etc., wherein LB and M9 are defined as those above.

In addition, since many factors can cause the vectors in the transformed host cells being unstable and lost, aspartase sequence fragment such as aspartase in E. Coli (SEQ ID:7) can be directly inserted into chromosome to solve the problem. Insertion can be achieved by phage/virus infection, transposition by transposons or homologous recombination. Therefore, one of the present invention's purposes is to provide a method for improving cell growth, which comprises steps as follows: (a) inserting the nucleic acid sequence of aspartase into the chromosome of a host cell to develop a recombinant host cell; and (b) culturing the recombinant host cell from step (a) in the medium to express the aspartase gene and improve the growth of said recombinant host cell. The aspartase nucleic acid sequence in step (a) comes from E. coli, bacteria, yeasts, fungi, insects, plants, animals or human cells. Wherein host cell in step (a) contains bacteria, yeasts, fungi, plant cells, insect cells or mammalian cells, preferably E. coli. Wherein medium in step (b) includes aspartate in the medium, for example, LB, LB with glucose, M9 with glucose and aspartate or M9 with glucose, yeast extract and aspartate etc., wherein LB and M9 are defined as those above.

The present invention further provides a method to increase expression and production of the target gene, which comprises the steps as follows: (a) constructing a recombinant vector containing aspartase nucleic acid sequence and desired target gene; (b) co-transforming the recombinant vector and aspartase nucleic acid sequence in step (a) to a host cell to form a recombinant host cell; and (c) culturing the recombinant host cell from step (b) in the medium and inducing said recombinant host cell to express aspartase and desired target gene, wherein said aspartase can increase the production of desired target gene in said recombinant host cell. The aspartase nucleic acid sequence in step (b) comes from E. coli, bacteria, yeasts, fungi, insects, plants, animals or human cells, preferably from E. coli. Wherein target gene products in step (a) include recombinant proteins or peptides. Said recombinant proteins comprise heterologous proteins and homologous proteins, like Aequorea green fluorescent protein, or homologous proteins, like β-galactosidase. Wherein host cell in step (b) include bacteria, yeasts, plant cells, insect cells or mammalian cells. Wherein medium in said step (c) contains aspartate in it, like LB, LB with glucose, M9 with glucose and aspartate or M9 with glucose, yeast extract, and aspartate etc., wherein LB and M9 are defined as those above.

The present invention also provides another method to increase target gene expression and production in cell, which comprises steps as follows: (a) inserting the aspartase nucleic acid sequence into a host cell chromosome to form a recombinant host cell; and (b) constructing a recombinant vector containing desired target gene; (c) transforming the recombinant vector in step (b) into host cell in step (a) to form a recombinant host cell; and (d) culturing the recombinant cell from step (c) in the medium and inducing the expression of aspartase and desired target gene in said recombinant host cell, wherein said aspartase can increase the production of desired target gene in said recombinant host cell. Recombinant proteins in previous said steps are homologous proteins, like Aequorea green fluorescent protein, or homologous proteins, like β-galactosidase. Host cells in said step (a) include bacteria, yeasts, Fungi, plant cells, insect cells or mammalian cells. Wherein medium in said step (d) contains aspartate in it, like LB, LB with glucose, M9 with glucose and aspartate or M9 with glucose, yeast extract and aspartate etc., wherein LB and M9 are defined as those above.

The present invention confers on the cell with aspartase (or aspartate ammonia-lyase) activity to transport the extracellular aspartate into cells and then convert it to fumarate, an intermediate of TCA cycle (See FIG. 1). In aerobic condition, the production level of aspartase is very low and its expression is subject to catabolite repression exerted by glucose (Halpern and Umbarger, (1960) J. Bacteriol. 80:285-288; Woods and Guest, (1987) FEMS Microbiol. Lett. 48:219-224). Therefore, the present invention constructs a vector with aspartase gene and expressing it in host cell to produce aspartase. After the transformation of the vector into host cell, the cell will then gain aspartase activity. According to applicants' research, the host producer cell endowed with aspartase activity can improve its characteristics in protein production. These include the increase in final cell density and yield of recombinant protein under an aerobic condition as a consequence of the improved flux distribution in metabolic pathways of cells. This result is particularly beneficial for the manipulation of the transformed host cell with high cell density. From the experiment results, the strategy can solve problems encountered in overproduction of recombinant proteins, and will have significant contribution to the field of bio-industry.

### BRIEF DESCRIPTION OF THE DRAWINGS

FIG. 1 shows the central metabolism in E. coli, which includes glycolysis and tricarboxylic acid cycle pathway. In the figure, ppc is phosphoenolpyruvate carboxylase gene; aspA is aspartase gene; NADH is nictinamide-adenine dinucleotide.

FIG. 2 is the construction map of plasmid pA199A-2. Wherein, it contains: pMB1 ori, origin of replication of plasmid pMB1; PA1, T7 A1 promoter; lacI, lacI protein repression gene; rrnBT1T2, rrnB transcriptional termination site; Ap^{r}, anti-ampicillin gene; and MCS, multiple cloning site where ribosome binding site (RBS) and restriction enzyme cleavage site are marked in MCS frame.

FIG. 3 is the construction map of plasmid pA1-AspA. Wherein, it contains: pMB1ori, origin of replication of plasmid pMB1; PA1, T7 A1 promoter; lacI, lacI protein repression gene; rrnBT1T2, rrnB transcriptional termination site; Ap^{r}, anti-ampicillin gene; aspA, aspartase structural gene; BamHI, restriction enzyme BamHI cleavage site; HindIII, restriction enzyme HindIII cleavage site; NruI, restriction enzyme NruI cleavage site.

FIG. 4 is the construction map of plasmid pACYC184. Wherein, it contains: Cm^{r}, anti-chloramphenicol gene; p15A ori, origin of replication of plasmid p15A; PA1, Tc^{r}, anti-tetracycline gene; NruI, restriction enzyme NruI cleavage site; HindIII, restriction enzyme HindIII cleavage site.

FIG. 5 is the construction map of pACYC-AspA. Wherein, it contains: Cm^{r}, anti-chloramphenicol gene; p15A ori, origin of replication of plasmid p15A; P_{A1}, T7 A1 promoter; aspA, aspartase structural gene; lacI, lacI protein repression gene; BamHI, restriction enzyme BamHI cleavage site; NruI, restriction enzyme NruI cleavage site; HindIII, restriction enzyme HindIII cleavage site.

FIG. 6 is the protein electrophoresis result of the aspartase-producing E. coli strain.

FIG. 7 is the growth curve of recombinant strains VJS676/pA199A-2 and VJS676/pA1-AspA in M9 with glucose medium, and M9 with glucose plus aspartate medium. 300 μM IPTG is added for induction (indicated by an arrow).

FIG. 8 is the growth curve of recombinant strains VJS676/pA199A-2 and VJS676/pA1-AspA in LB medium. 300 μM IPTG is added for induction (indicated by an arrow).

FIG. 9 is the construction map of plasmid pAH55. Wherein, it contains: Km^{r}, anti-kanamycin gene; R6Kori, origin of replication of plasmid R6K; Ptac, tac promoter; lacI^{q}, lacI protein repression gene; t0, A phage transcriptional termination site.

FIG. 10 is the construction map of plasmid pTrc99A. Wherein, it contains: Ap^{r}, anti-ampicillin gene; Ptrc, trc promoter; lacI^{q}, lacI protein repression gene; rrnBT1T2, rrnB transcriptional termination site; EcoRV, restriction enzyme EcoRV cleavage site; and MCS, multiple cloning site where restriction enzyme cleavage site is marked in MCS frame.

FIG. 11 is the construction map of plasmid pTac99A. Wherein, it contains: Ap^{r}, anti-ampicillin gene; Ptac, tac promoter; lacI^{q}, lacI protein repression gene; rrnBT1T2, rrnB transcriptional termination site; EcoRV, restriction enzyme EcoRV cleavage site; t0, λ phage transcriptional termination site; and MCS, multiple cloning site where restriction enzyme cleavage site is marked in MCS frame.

FIG. 12 is the construction map of plasmid pTac-Z. Wherein, it contains: Ap^{r}, anti-ampicillin gene; Ptac, tac promoter; lacI^{q}, lacI protein repression gene; rrnBT1T2, rrnB transcriptional termination site; lacZ, β-galactosidase structural gene; HindIII, restriction enzyme HindIII cleavage site; NcoI, restriction enzyme NcoI cleavage site; to, λ phage transcriptional termination site.

FIG. 13 is the construction of plasmid pACYC-A1. Wherein, it contains: Cm^{r}, anti-chloramphenicol gene; p15A ori, origin of replication of plasmid p15A; P_{A1}, T7 A1 promoter; lacI, lacI protein repression gene; NruI, restriction enzyme NruI cleavage site; [BamHI/HindIII], blunt end of BamHI, HindIII cleavage sites formed during plasmid construction.

FIG. 14 is the construction map of plasmid pGFPuv. Wherein, it contains Ap^{r}, anti-ampicillin gene; pUC ori, origin of replication of plasmid pUC; P_{lac}, lac promoter; GFPuv; Aequorea green fluorescent protein structural gene.

FIG. 15 is the Western blot of Aequorea green fluorescent protein produced in E. coli with aspartase activity.

### DETAILED DESCRIPTION OF THE INVENTION

The present invention makes use of a vector containing aspartase gene or aspartase gene fragment in the host cell to regulate and produce aspartase. The present invention can endow host cell with aspartase activity, and further give the host cell the characteristics of increasing cell growth and improving the production of recombinant proteins.

The advantages of the present invention are further depicted with the illustration of examples. The following is a description of the exemplary case of carrying out the platelets provided by the invention for bioactivity testing. This exemplary case is not to be taken in a limiting sense, but is made merely for the purpose of further illustrating the materials and methods for practicing the present invention.

### EXAMPLE

Methods and Materials

The experimental protocols and DNA cloning technique used in the present invention refer to the textbook written by Sambrook J, Russell D W (2001) Molecular Cloning: a Laboratory Mannual, 3^{rd} ed. Cold Spring Harbor Laboratory Press, New York.

Techniques used in the present invention include DNA cleavage with restriction enzymes, DNA ligation with T4 DNA ligase, polymerase chain reaction (PCR), agarose gel electrophoresis, Western blotting, sodium dodecyl sulphate-polyacrylamide gel electrophoresis (SDS-PAGE) and plasmid transformation etc. Any person skilled in the art can carry out the techniques by his own profession without excessive experiments.

The competent cells used for plasmid transformation was treated with CaCl₂. Cell density was measured by spectrophotometer (V530, Jasco) at 550 nm wavelength, and the resulting optical density was recorded as OD₅₅₀. Protein concentration was analyzed with Protein Assay Reagent, Biorad Co., and Image analyzer (GAS9000, UVItec) was used to quantify proteins separated by electrophoresis.

Purification of bacteria chromosome, plasmids and DNA fragments were conducted by respectively using Wizard® Genomic DNA Purification Kit (Promega Co.), QIAprep Spin Miniprep Kit (Qiagen Co.) and NucleoSpin® Nucleic Acid Purification Kit (Clontech Co.) or other commercial purification kits.

All restriction enzymes, T4 ligase and Pfu DNA polymerase were bought from New England Biolabs. Primers used in the PCR were synthesized by Ming-Shin Tech. Co., Taipei. Primary antibody for detecting Aqeuorea green fluorescent protein was bought from BD Biosciences Clontech, and horseradish peroxidase-conjugated goat anti-rabbit IgG type secondary antibody and other chemical reagents were all bought from Sigma Chemical Co.

### Example 1

### Construction of Plasmids Containing Aspartase Structural Gene (aspA)

The intermediate cells used for DNA cloning in the present example were E. coli XLI-Blue (Stratagene Co.).The bacteria strain was cultured in Luria-Bertani (LB) medium (containing yeast extract (5 g/L), tryptone (10 g/L), NaCl (5 g/L)) under 37° C. (Miller, J. H. (1972), Experiments in Molecular Genetics, Cold Spring Harbor Laboratory, Cold Spring Harbor, N.Y.). Transformed E. coli were cultured in the LB with antibiotics, and the used antibiotics included ampicillin, chloramphenicol and kanamycin and the amount used were 50, 20 and 25 μg/ml, respectively.

A. Construction of the Recombinant Plasmid, pA1-AspA, Containing Aspartase Gene (aspA)

According to FIG. 3, high-copy-number plasmid pA1-AspA used in the example comprises a T7 A1 promoter, lacI repression gene and pMB1 origin of replication. Product of lacI repression gene can control T7 A1 promoter to regulate the expression of aspartase (EC 4.3.1.1). The construction is as follows:

First, the following two primers based on the nucleic acid sequence of the aspartase gene (SEQ ID NO: 7) were synthesized (Woods et al. (1986), Biochem. J. 237:547-557)

Forward primer

(SEQ ID NO: 1)

5′-cacaggatccacaacattcgtatcgaag-3′
Reverse primer

(SEQ ID NO: 2)

5′-gctaagcttactgttcgctttcatcattatagc-3′

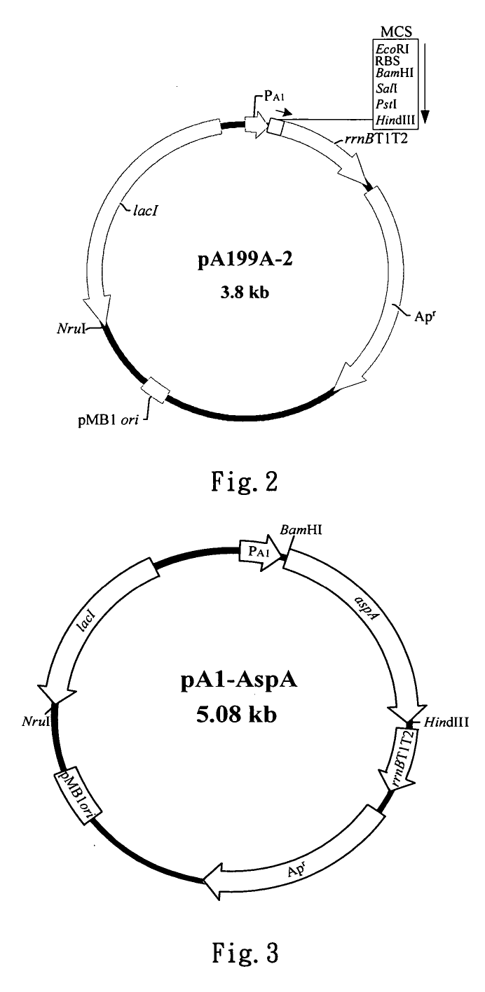

Aforementioned two primers (SEQ ID NO: 1 and SEQ ID NO: 2) were designed for restriction enzymes BamHI and HindIII cleavage (underlined) respectively. Then, E. coli strain VJS676 (Δ (argF-lac) U169) (from Dr. Stewart, University of California, Davis, Calif., USA) was purified by Wizard® Genomic DNA Purification Kit as template and was conducted PCR reaction with two primers mentioned above to amplify the DNA fragment of aspartase gene (1.5 kb). Further, the amplified DNA fragments were purified with NucleoSpin® Nucleic Acid Purification Kit; after the amplified DNA fragments were cleaved with restriction enzymes BamHI and HindIII, the cleaved products were incorporated into plasmid pA199A-2 cleaved by BamHI and HindIII (Shown in FIG. 2) (Chao et al. (2003), Biotechnol. Prog. 19: 1076-1080) to obtain plasmid pA1-AspA (shown in FIG. 3).

B. Construction of the Recombinant Plasmid, pACYC-AspA, Containing Aspartase Gene (aspA)

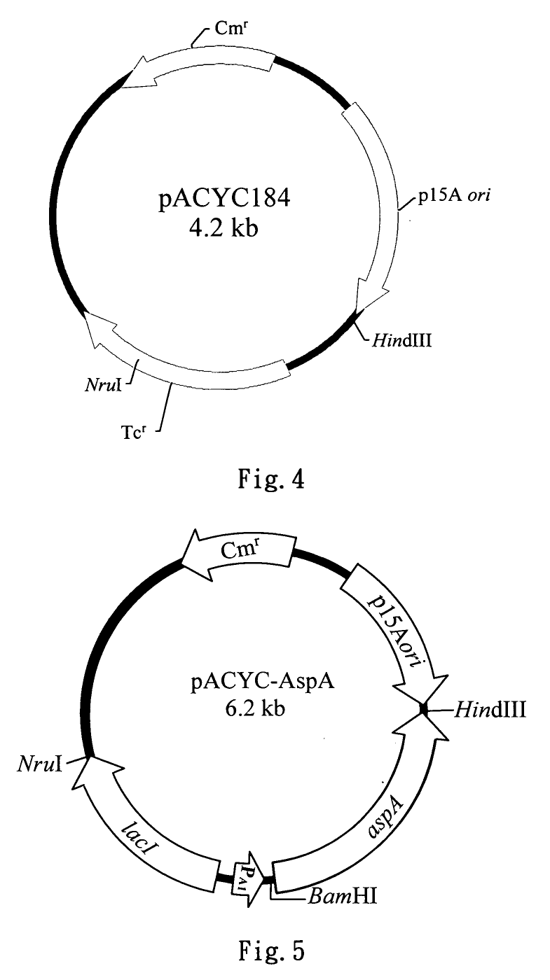

According to FIG. 5, low-copy-number plasmid pACYC-AspA used in the example comprises a T7 A1 promoter, lacI repression gene and p15A origin of replication. The construction is as follows: First, used restriction enzymes NruI and HindIII cleavage to get a DNA fragment with one lacI repression gene, T7 A1 promoter and aspartase gene; then, incorporated it into plasmid pCYC184 cleaved by NruI and HindIII (shown in FIG. 4) to get plasmid pACYC-AspA (shown in FIG. 5.).

### Example 2

### Production of Aspartase in E. coli

According to Methods and Materials, plasmids pA199A-2, pA1-AspA, pACYC184 and pACYC-AspA from example 1 were transformed into E. coli strain VJS676 and recombinant strains VJS676/pA199A-2, VJS676/pA1-AspA, VJS676/pACYC184 and VJS676/pACYC-AspA were obtained in the present example.

Colonies selected from agar plates were inoculated into 5 ml LB with 50 μg/ml ampicillin or 20 μg/ml chloramphenicol at 37° C. for overnight. Thereafter, the cultured cells were inoculated into a 250 ml flask with 25 ml LB medium (containing 50 μg/ml ampicillin or 20 μg/ml chloramphenicol). The initial cell density was set at 0.05 (OD₅₅₀), and the inoculated cells were cultured in a shaking incubator with 250 rpm at 37° C. Upon cell density reaches 0.3 (OD₅₅₀), different concentrations of IPTG were added into culture medium to induce recombinant strain for the production of recombinant proteins. The cell growth was monitored along the time course. 6 hours after adding inducer, the cells were collected by centrifugation and disrupted with French Press (Thermo Spectronic). Subsequently, cell debris was removed by centrifugation and the supernatant was collected to determine the protein concentration using Protein Assay Reagent (Biorad Co.). Finally, the collected protein samples (containing 20 μg protein) were loaded onto 12% polyacrylamide gel to perform SDS-PAGE analysis.

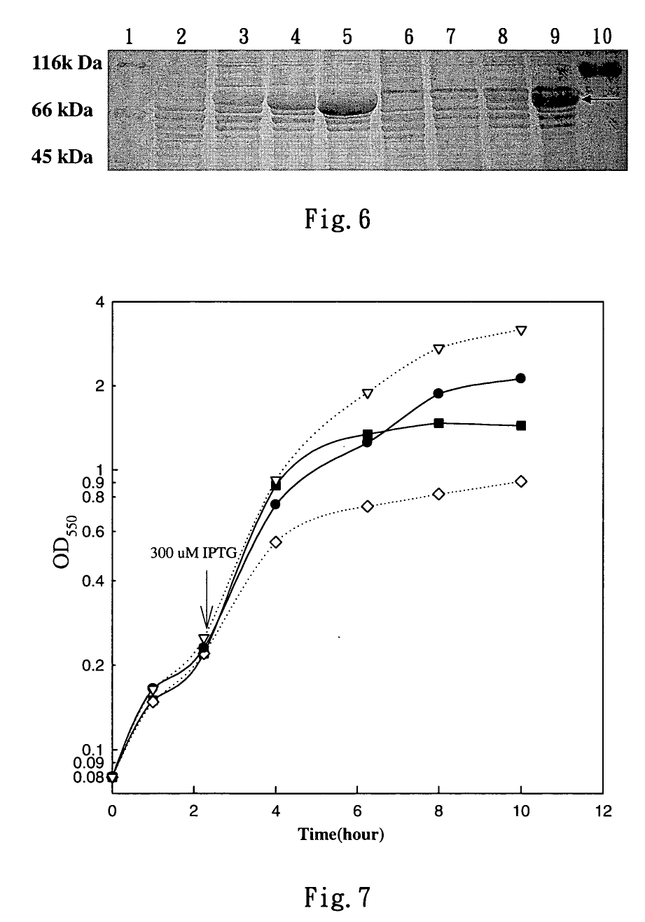

According to electrophoresis result shown in FIG. 6, after fermentation for 10 hours, the aspartase production of induced recombinant bacteria strain is as follows: lane 1: protein standard (MBI Fermentas); lane 2: uninduced recombinant strain (VJS676/pA1-AspA); lane 3: 50 μM IPTG-induced recombinant strain (VJS676/pA1-AspA); lane 4: 100 μM IPTG-induced recombinant strain (VJS676/pA1-AspA); lane 5: 300 μM IPTG-induced recombinant strain (VJS676/pA1-AspA); lane 6: control strain with no aspartase production (VJS676/pACYC-AspA); lane 7: 50 μM IPTG-induced recombinant strain (VJS676/pACYC-AspA); lane 8: 100 μM IPTG-induced recombinant strain (VJS676/pACYC-AspA); lane 9: 300 μM IPTG-induced recombinant strain (VJS676/pACYC-AspA); lane 10: 2 μg Bovine Serum Albumin, BSA. Before IPTG induction, high-copy-number plasmid pA1-AspA (recombinant strain VJS676/pA1-AspA) and low-copy-number plasmid pACYC-AspA (recombinant strain VJS676/PACYC-AspA) both produce little amount of aspartase. On the contrary, after IPTG induction, recombinant strain started to produce and accumulate large amount of aspartase, and the amount of enzyme production was proportional to the inducer (IPTG) concentration.

The assay of aspartase activity in E. coli was carried out based on the method described in our previous paper (Chao et al. (2000) Enzyme Microb. Technol., 27:19-25). Mainly, collected protein sample was added (0.05 mg) into 1-ml reaction solution consisting of 100 mM aspartase, 100 mM Tris buffer (pH 8.4) and 5 mM MgSO₄. After reacting at room temperature for 10 minutes, the product was analyzed with HPLC. The analyzing condition was as follows: 0.05N sulphuric acid as mobile phase solution; flow rate: 0.5 mL/min; detection wave length: 210 nm. The unit of enzyme specific activity is U/mg, and the unit of enzyme activity (U) is defined as per μ mole of product produced per min From the measured enzyme activity shown in Table 1, adding different concentrations of IPTG into culture medium can induce various levels of aspartase in recombinant strains VJS676/pA1-AspA and VJS676/pACYC-AspA, and the production of aspartase corresponded postively with an increasing concentration of IPTG. It was estimated that 50- and 100-fold of aspartase activity could be produced upon induction relative to the uninduced level.

Taking the results from FIG. 6 and Table 1, the transformed E. coli strain in the present invention is able to produce aspartase, and the protein production is controlled by different IPTG concentrations.

TABLE 1
Activity of aspartase produced in recombinant
bacteria strain induced by IPTG

| | VJS676/pA1-AspA | VJS676/pACYC-AspA |
|---|---|---|
| IPTG (μM) | (U/mg) | (U/mg) |
| 0 | 0.39 | 0.13 |
| 50 | 11.62 | 1.97 |
| 100 | 17.18 | 6.23 |
| 300 | 20.22 | 13.38 |

### Example 3

### Production of Aspartase to Improve Cell Growth

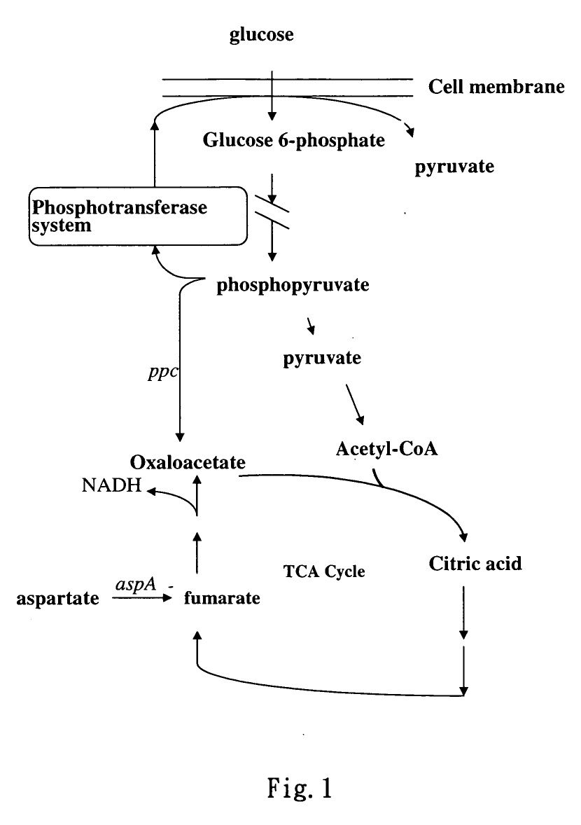

In the control experiment of present invention, the culture method for recombinant strains VJS676/pA199A-2 and VJS676/pA1-AspA was the same as that in example 2. The medium used is M9 (Miller, J. H. (1972) Experiments in Molecular Genetics, Cold Spring Harbor Laboratory, Cold Spring Harbor, N.Y.) plus glucose (0.1%) and ampicillin (15 μg/mL). The medium M9 contains Na₂HPO₄ (6 g/L), KH₂PO₄ (3 g/L), NaCl (0.5 g/L), NH₄Cl (1 g/L), MgSO₄·7H₂O (1 mM) and CaCl₂ (0.1 mM). When the cell density of freshly inoculated bacteria achieved around 0.3 (OD₅₅₀), 300 μM IPTG was added into culture medium to induce the production of recombinant proteins in the cells, and cell density was measured along with time. From the growth curve in FIG. 7, after IPTG induction, growth of the aspartase-producing strain VJS676/pA1-AspA (⋄) became slow. The cell density reached only 0.9 (OD₅₅₀) after fermentation for 10 hours. However, during the same fermentation period, cell density of non-aspartase-producing strain VJS676/pA199A-2 (▪) was 1.5 (OD₅₅₀). According to the function of aspartase, the enzyme primarily decomposes aspartate into fumarate and NH₄⁺ (See FIG. 1). Under this culturing condition, aspartase induced in E. coli doesn't have obvious physiological function. Hence, FIG. 7 shows that the production of recombinant protein like aspartase could impose metabolic burden on cell, thereby inhibiting the growth of cell.

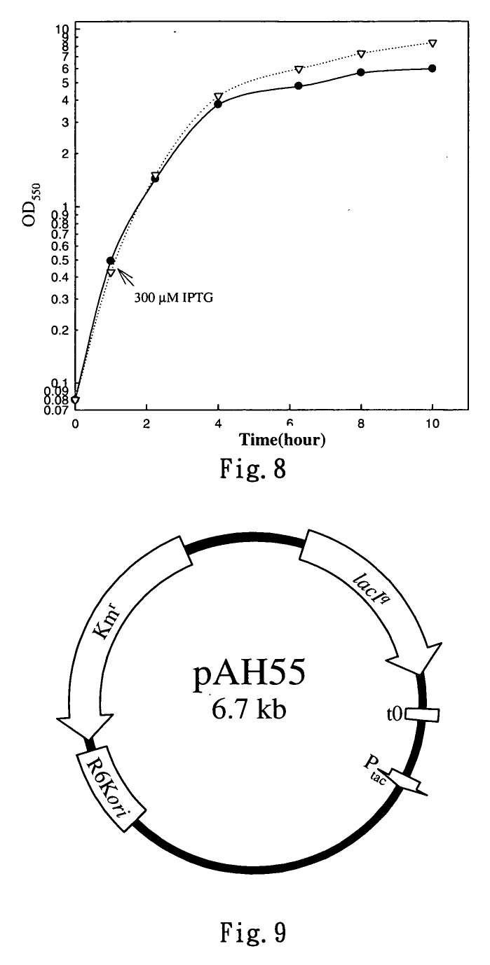

The experimental group in the present example used M9 with glucose (0.1%), ampicillin (15 μg/mL) and aspartate (0.5%) as medium. The culture method of recombinant strains VJS676/pA199A-2 and VJS676/pA1-AspA was the same as that in previous procedure. Again, while cell density of the freshly inoculated bacteria achieved around 0.3 (OD₅₅₀), 300 μM IPTG was added into culture medium to induce the production of recombinant protein, and cell density was measured along with time. From FIG. 7, after fermentation for 10 hours, cell density of non-aspartase-producing strain VJS676/pA199A-2 (●) was 2.1 (OD₅₅₀), and its specific growth rate was 0.23h⁻¹; in contrast, aspartase-producing recombinant strain VJS676/pA1-AspA (∇)grew faster (its specific growth rate was 0.32 h⁻¹), and its final cell density could reach 3.2 (OD₅₅₀). The result shows that the presence of aspartate in medium, the induction of aspartase expression in E. coli render the host cells grow better. In this case, the active function of aspartase is responsible for the decomposition of aspartate into fumarate. Therefore, additional carbon source rather than glucose enters TCA cycle, and fumarate is further converted to OAA with the concomitant generation of NADH via the functioning of TCA cycle. Finally, OAA combines with acetyl-CoA from glycolysis to produce citric acid (See FIG. 1). As compared with non-aspartase-producing strain, aspartase-producing strain can decompose extracellular aspartate to increase more carbon sources, TCA cycle intermediates, and energy. This not only results in the alleviation of the metabolic burden exerted by aspartase production (shown in FIG. 7), but increase the specific growth rate of cell by 40% more and increase the cell density by 60% more. The present example further used LB with ampicillin (50 μg/mL) as medium to culture recombinant strains VJS676/pA199A-2 and VJS676/pA1-AspA with the same method as said previously. While cell density of the freshly inoculated bacteria achieved 0.3 (OD₅₅₀), 300 μM IPTG was added into the culture medium to induce the production of recombinant protein, and cell density was measured along with time. As shown in FIG. 8, after fermentation for 10 hours, cell density of the aspartase-producing strain VJS676/pA1-AspA (∇) was 8.4 (OD₅₅₀), and cell density of non-aspartase-producing strain VJS676/pA199A-2 (●) was 6.0 (OD₅₅₀). Since LB medium contains tryptone and yeast extract composition, aspartate is expected to be present in the medium (similar to medium containing aspartate used in experimental group in FIG. 7). Consequently, aspartase-producing strain can decompose extracellular aspartate to have more carbon source and energy to increase cell density by 55% more.

In summary, the activity of aspartase endows transformed host cell with the ability to improve cell growth and increase cell density.

### Example 4

### Production of Homologous Protein—β-Galactosidase in E. coli Containing Aspartase Activity

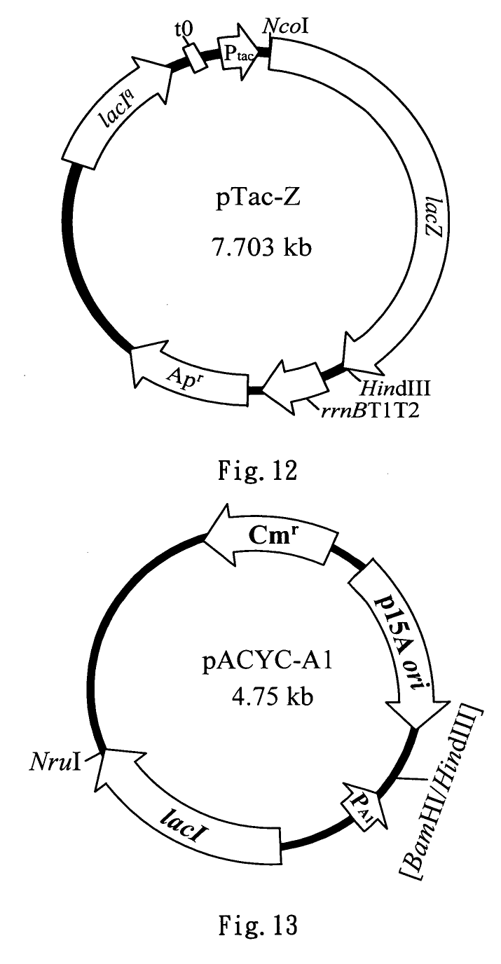

According to FIG. 12, the plasmid pTac-Z used in present example contained a tac promoter and lacI^{q} repression gene, and its construction was as follows: First, two primers based on nucleic acid sequence of plasmid pAH55 (as shown in FIG. 9) (Haldimann and Wanner, (2001), J. Bacteriol., 183:6384-6393.) were synthesized.

| | |
|---|---|
| Forward primer | |
| 5′-taactcgcgataattgcgttgcgctcac-3′ | (SEQ ID NO: 3) |
| Reverse primer | |
| 5′-cgcccatggtatatctccttcttacaagc-3′ | (SEQ ID NO: 4) |

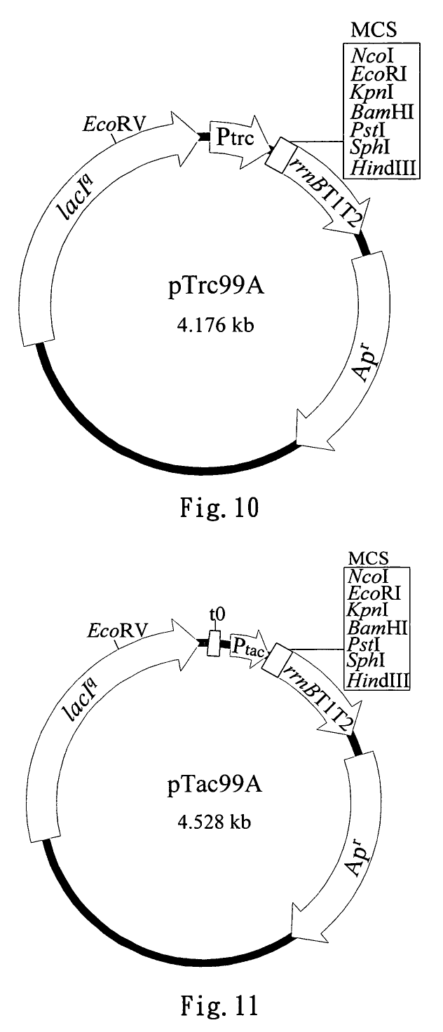

The reverse primer was designed to have restriction enzyme NcoI cleavage site (underlined). Then, the PCR template—plasmid pAH55 was purified by QIAprep® Spin Miniperp kit; templates were added with two said primers to conduct the PCR reaction to amplify a DNA fragment (1.8 kb) containing lacI^{q} repression gene and tac promoter. Afterwards, the amplified DNA fragment was purified by NucleoSpin® Nucleic Acid Purification Kit and was cut with restriction enzyme NcoI/EcoRV, and the cleavage product were incorporated into plasmid pTrc99A cut with NcoI/EcoRV (as shown in FIG. 10) (Amann et al., (1988), Gene, 69:301-315) to get plasmid pTac99A (as shown in FIG. 11).

Furthermore, synthesize the following two primers based on nucleic acid sequence of β-galactosidase gene:

| | |
|---|---|
| Forward primer | |
| 5′-acagccatggccatgattacggattcac-3′ | (SEQ ID NO: 5) |
| Reverse primer | |
| 5′-cggaagcttttatttttgacaccagacc-3′ | (SEQ ID NO: 6) |

Two primers described above were designed to have restriction enzymes NcoI and HindIII cleavage sites (underlined). Subsequently, wild type E. coli W3110 purified by Wizard® Genomic DNA Purification Kit was used as template, and the PCR reaction was carried out with primers of SEQ ID: 5 and 6 to amplify a DNA fragment containing β-galactosidase gene (lacZ) (3 kb). After that, the amplified DNA fragment was purified by NucleoSpin® Nucleic Acid Purification Kit and was cut with restriction enzyme NcoI and HindIII; the cleavage product was incorporated into plasmid pTrc99A cut with NcoI and HindIII to get plasmid pTac-Z (as shown in FIG. 12).

Based on Methods and Materials, plasmid pTac-Z and plasmid pACYC184 or pACYC-AspA were co-transformed into E. coli strain VJS676 to obtain recombinant strains VJS676/pACYC184/pTac-Z and VJS676/pACYC-AspA/pTac-Z, respectively.

Culturing of recombinant strain proceeds with the method described in example 2. The media used were LB, LB with glucose (0.2%), M9 with glucose (0.1%) and yeast extract (0.5%), and M9 with glucose (0.1%), yeast extract (0.2%) and aspartate (0.5%), respectively; antibiotics used were 15 μg/mL ampicillin and 10 μg/mL chloramphenicol. While the cell density of freshly inoculated bacteria achieved around 0.3 (OD₅₅₀), 100 and 300 μM IPTG were added into culture medium to induce the production of recombinant protein, and cell culture was sampled along with time for the measurement of cell density. After culturing for 10 hours, cells were collected with centrifugation and the activity of β-galactosidase was measured.

Measurement of β-galactosidase activity in E. coli was based on the method described in Miller, J. H. (1972) Experiments in Molecular Genetics, Cold Spring Harbor, N.Y.: Cold Spring Harbor Laboratory. 0.1 mL of cell culture was taken and mixed with 0.9 mL Z buffer (16.1 g/L Na₂HPO₄·7H₂O, 5.5 g/L NaH₂PO₄·H₂O, 0.75 g/L KCl, 0.246 g/L MgSO₄ ·7H₂O, 2.7 mL β-mercaptoethanol), and the final volume was kept 1 ml. Afterwards, 10 μL toluene was added, the cells were broken by vigorous vortex, followed by centrifugation, and the supernatant was collected.

Add 0.2 mL reaction solution containing o-nitrophenyl-β-D-thiogalactoside (4 mg/mL) into the collected supernatant. After reacting at room temperature for 5 minutes, 0.5 mL Na₂Co₃ (1M) was added to stop the reaction and detected with 420 nm wave length. Unit of specific activity of β-galactosidase is defined as Miller unit, and the total enzyme activity is the result of the multiplication of enzyme specific activity with cell density (OD₅₅₀).

TABLE 2
The cell density and (β-galactosidase production
of IPTG-induced recombinant strains

| | | | | Enzyme | | |
|---|---|---|---|---|---|---|
| | | IPTG | Final | Specific | Enzyme | |
| | | Induction | Cell | Activity | Total | |
| Strain VJS676 | Nutrition* | Concentration | Density | (Miller | Activity* | |
| Plasmid | Base | (μM) | (OD₅₅₀) | unit) | (Unit) | |
| pACYC184/pTac-Z | LB | 0 | 5.1 | 330 | 1690 | |
| | | 100 | 4.8 | 26600 | 127680 | (1) |
| | | 300 | 4.7 | 29100 | 136770 | (1) |
| pACYC-AspA/pTac-Z | LB | 0 | 5.0 | 310 | 1550 | |
| | | 100 | 5.7 | 29810 | 169920 | (1.3) |
| | | 300 | 5.1 | 33800 | 172380 | (1.3) |
| pACYC184/pTac-Z | LBG | 0 | 6.0 | 130 | 780 | |
| | | 100 | 5.9 | 24600 | 145140 | (1) |
| | | 300 | 5.8 | 29800 | 172840 | (1) |
| pACYC-AspA/pTac-Z | LBG | 0 | 5.7 | 300 | 1710 | |
| | | 100 | 6.5 | 38800 | 252200 | (1.7) |
| | | 300 | 5.4 | 58300 | 314820 | (1.8) |
| pACYC184/pTac-Z | M9Y | 0 | 5.6 | 500 | 2800 | |
| | | 100 | 5.2 | 16600 | 86320 | (1) |
| | | 300 | 4.8 | 18000 | 86400 | (1) |
| pACYC-AspA/pTac-Z | M9Y | 0 | 5.7 | 860 | 4900 | |
| | | 100 | 5.8 | 18100 | 104980 | (1.2) |
| | | 300 | 5.2 | 24000 | 124800 | (1.4) |
| pACYC184/pTac-Z | M9YA | 0 | 4.3 | 390 | 1680 | |
| | | 100 | 4.1 | 23800 | 97580 | (1) |
| | | 300 | 3.9 | 27100 | 105690 | (1) |
| pACYC-AspA/pTac-Z | M9YA | 0 | 4.1 | 380 | 1560 | |
| | | 100 | 5.2 | 42800 | 222560 | (2.3) |
| | | 300 | 4.4 | 58700 | 258280 | (2.4) |

*Note:
Numbers in the parentheses indicate the ratio of total enzyme activity of recombinant strain (VJS676/pACYC-AspA/pTac-Z) to that of recombinant strain (VJS676/pACYC184/pTac-Z) in the same conditions.

These are calculated by dividing the total enzyme activity of each recombinant strain (VJS676/pACYC-AspA/pTac-Z) receiving IPTG induction by that of control strain (VJS676/pACYC184/pTac-Z) induced by the same IPTG concentration. Nutrition base: LB is LB medium; LBG is LB medium with glucose (0.2%); M9Y is M9 medium with glucose (0.1%) and yeast extract (0.5%); M9YA is M9 medium with glucose (0.1%), yeast extract (0.2%) and aspartate (0.5%).

Table 2 shows that, while cultured in different media, the production of β-galactosidase from control strain (VJS676/pACYC184/pTac-Z) and aspartase-producing strain (VJS676/pACYC-AspA/pTac-Z) is proportional to the induction amount of IPTG. In LB medium with the same induction condition, the total β-galactosidase production (total enzyme activity) in the aspartase-producing strain increases 30% more relative to that in the control strain. Meanwhile, in LB with glucose (LBG) with the same induction condition, the total β-galactosidase production in the aspartase-producing strain increases by 70-80%. As described in example 3, since LB medium contains aspartate, the aspartase-producing strain can gain more carbon source, TCA cycle intermediates, and energy by the act of aspartase activity. Similarly, while cultured in M9 medium with glucose and yeast extract (M9Y), in comparison with that of induced control strain, the total β-galactosidase production in the bacteria strain with aspartase activity can increase 30-50%. However, while cultured in M9 medium with glucose, yeast extract and aspartate (M9YA), the total β-galactosidase production in the bacteria strain with aspartase activity can increase 130-140% more than that in the induced control strain. The results indicate that the presence of extra aspartate in the medium, more carbon source, TCA cycle intermediates, and energy can be produced in the cell with aspartase activity; hence, production of recombinant protein can be further improved.

### Example 5

### Production of Heterologous Protein—Aequorea Green Fluorescent Protein in E. coli Containing Aspartase Activity

According to FIG. 13, the structure of plasmid pACYC-A1 used in present example is similar to that of plasmid pACYC-AspA (See FIG. 5). However, plasmid pACYC-Al doesn't contain aspA structural gene. The plasmid is constructed by cutting pACYC-AspA with restriction enzyme BamHI and HindIII to remove aspA structural gene. The cleavage sites were blunted with T4 DNA polymerase and ligated with T4 DNA ligase to obtain plasmid pACYC-A1 (See FIG. 13).

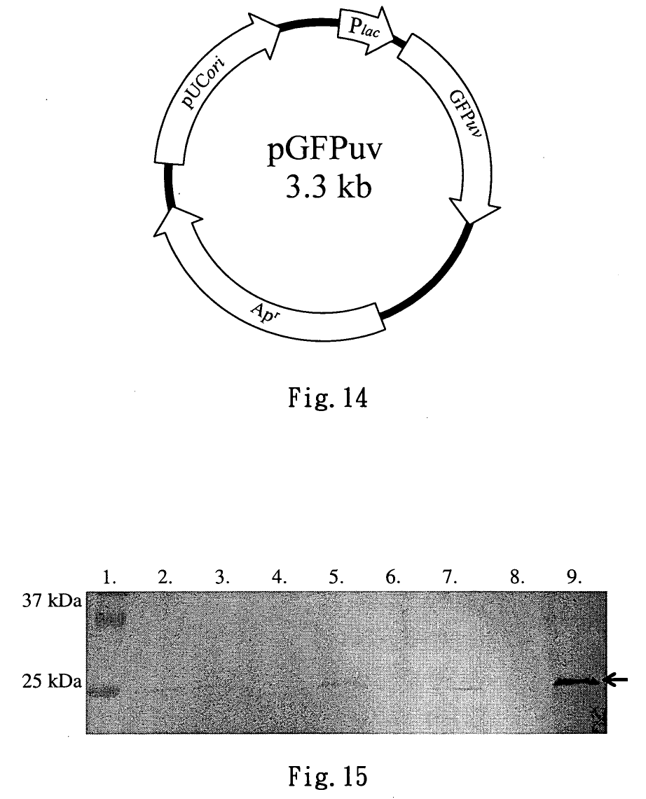

As shown in FIG. 14, plasmid pGFPuv (from BD, Biosciences Clontech) contains pUC origin of replication, ampicillin-resistant gene and a mutant Aequorea Victoria green fluorescent protein structural gene, and the expression of the structural gene is controlled by a lac promoter. Based on Methods and Materials, co-transformation of plasmids pACYC-A1 and pACYC-AspA with plasmid pGFPuv into E. coli strain BL21 (Novagen Co.) was performed to obtain recombinant strains BL21/pGFPuv/pACYC-A1 and BL21/pGFPuv/pACYC-AspA, respectively.

Culture of recombinant strains is based on procedures described in example 2. The nutrition bases used are LB with glucose (0.2%) and M9 with glucose (0.1%), yeast extract (0.2%) and aspartate (0.5%), respectively. The amounts of used antibiotics are 15 μg/mL ampicillin and 10 μg/mL chloramphenicol, respectively. While cell density of freshly inoculated bacteria reached around 0.3 (OD₅₅₀), 100 μM IPTG was added into the culture medium for the production of recombinant protein, and cell culture was sampled along with the time for the measurement of cell density. After culturing for 10 hours, cells were collected with centrifugation, disrupted by French Press, and then centrifuged to recover cell supernatant. Afterwards, protein concentration in the collected supernatant was measured by Protein Assay Reagent, BioRad Co., and the production of recombinant protein was assayed by Western blotting.

According to Western blot in FIG. 15, it shows the production of Aequorea green fluorescent protein in the aspartase-strain (BL21/pGFPuv/pACYC-AspA) and the control strain (BL21/pGFPuv/pACYC-A1) by immuno assay with primary antibody against Aequorea green fluorescent protein in M9 with 0.1% glucose, 0.2% yeast extract and 0.5% aspartate (lane 2-5), and LB with 0.1% glucose (lane 6-9). Wherein lane 1 is protein standard, MBI Fermentas; lane 2 is uninduced control strain BL21/pGFPuv/pACYC-A1; lane 3 is the control strain BL21/pGFPuv/pACYC-A1 induced by 100 μM IPTG; lane 4 is uninduced aspartase-producing strain BL21/pGFPuv/pACYC-AspA; lane 5 is the aspartase-producing strain BL21/pGFPuv/pACYC-AspA induced by 100 μM IPTG; lane 6 is uninduced control strain BL21/pGFPuv/pACYC-A1; lane 7 is the control strain BL21/pGFPuv/pACYC-A1 induced by 100 μM IPTG; lane 8 is uninduced aspartase-producing strain BL21/pGFPuv/pACYC-AspA; lane 9 is aspartase-producing strain BL21/pGFPuv/pACYC-AspA induced by 100 μM IPTG.

While using M9 with glucose, yeast extract and aspartate as medium, after fermentation for 10 hours, cell density of uninduced control strain BL21/pGFPuv/pACYC-A1 and recombinant strain BL21/pGFPuv/pACYC-AspA can reach 4.0 (OD₅₅₀). On the other hand, after IPTG induction, cell density of the control strain BL21/pGFPuv/pACYC-A1 can be 3.6 (OD₅₅₀), and that of strain BL21/pGFPuv/pACYC-AspA having aspartase activity can reach 4.6 (OD₅₅₀). According to Western blotting shown in FIG. 15, IPTG-induced strains can produce more Aequorea green fluorescent proteins. Moreover, the result by the assay with Image analyzer (GAS9000, UVItec) shows that, in comparison with induced control strain (lane 3), induced strain with aspartase activity can produce 100% more Aequorea green fluorescent proteins (lane 5).

While culturing in LB plus glucose medium, after fermentation for 10 hours, cell density of the control strain BL21/pGFPuv/pACYC-A1 is 5.1 (OD₅₅₀), irrespective of IPTG. And, cell density of uninduced recombinant strain BL21/pGFPuv/pACYC-AspA can reach 5.1 (OD₅₅₀). After IPTG induction, cell density of strain BL21/pGFPuv/pACYC-AspA with aspartase activity can be 6.4 (OD₅₅₀). Likewise, based on Western blot in FIG. 15, the production of Aequorea green fluorescent protein in the control strain BL21/pGFPuv/pACYC-A1 and recombinant strain BL21/pGFPuv/pACYC-AspA can be increased by IPTG induction. The assay by Image analyzer (GAS9000, UVItec) shows that, as compared to induced control strain (lane 7), induced strain with aspartase activity can produce 5 times more Aequorea green fluorescent proteins (lane 5).

In summary, no mater using M9 with glucose, yeast extract and aspartate as medium or using LB with glucose as medium, strain with aspartase activity (BL21/pGFPuv/pACYC-AspA) can produce 1 to 5 times more amount of green fluorescent protein relative to induced control strain (BL21/pGFPuv/pACYC-A1) under the same induction condition. In addition, the growth of the former is better and the final cell density can be increased by 20% or more. The example indicates that aspartase in the present invention can improve the production of recombinant protein, such as Aequorea green fluorescent protein, by the transformed host cells.

### Other Embodiments

All features disclosed herein may be combined in any form with other methods and replaced by other features with identical, equivalent or similar purpose. Thus except for the part that is specifically emphasized, all features disclosed herein constitute only one embodiment among the numerous equivalent or similar features.

All modifications and alterations to the descriptions disclosed herein made by those skilled in the art without departing from the spirits of the invention and appended claims shall remain within the protected scope and claims of the invention.

What is claimed is:

1. A method to promote cell growth, comprising:
(a) cloning one or more aspartase coding sequence into a vector to become a recombinant vector, wherein said recombinant vector including a promoter and one or more aspartase coding sequences manipulated to link and insert into the downstream of said promoter;
(b) transforming said recombinant vector into host cells and producing recombinant host cells; and
(c) culturing said host cells in culture medium and making said cells to express aspartase, then promoting said recombinant host cells growth.

2. The method as claimed in claim 1, wherein said promoter in step (a) is an IPTG-induced promoter which is used to regulate the expression of the aspartase gene.

3. The method as claimed in claim 1, wherein said promoter in step (a) is constitutive promoter or any other regulating promoter.

4. The method as claimed in claim 1, wherein said aspartase coding sequences in step (a) are from E. coli, bacteria, yeast, fungus, insect, plant, animal or human.

5. The method as claimed in claim 1, wherein said aspartase coding sequences in step (a) are from E. coli.

6. The method as claimed in claim 1, wherein the host cells in step (b) are from E. coli, bacteria, yeast, fungus, plant cells, insect cells or mammal cells.

7. The method as claimed in claim 1, wherein the culture medium in step (c) contains aspartate.

8. The method as claimed in claim 7, wherein said medium is LB.

9. The method as claimed in claim 7, wherein said medium contains LB and glucose.

10. The method as claimed in claim 7, wherein said culture medium contains M9, glucose, and aspartate, wherein said M9 contains Na₂HPO₄ (6 g/L), KH₂PO₄ (3 g/L), NaCl (0.5 g/L), NH₄Cl (1 g/L), MgSO₄·7H₂O (1 mM), CaCl₂ (0.1 mM).

11. The method as claimed in claim 7, wherein said culture medium contains M9, glucose, yeast extract and aspartate, wherein said M9 contains Na₂HPO₄ (6 g/L), KH₂PO₄ (3 g/L), NaCl (0.5 g/L), NH₄Cl (1 g/L), MgSO₄·7H₂O (1 mM), CaCl₂ (0.1 mM).

12. A method to increase the amount of the target gene expression, comprising:
(a) constructing a recombinant vector containing aspartase coding sequence, wherein said recombinant vector including a promoter and one or more aspartase coding sequences manipulated to link and insert into the downstream of the promoter;
(b) constructing a recombinant vector containing target gene to be expressed;
(c) co-transforming said recombinant vector in step (a) and in step (b) into host cells and producing recombinant host cells; and
(d) culturing said host cells in culture medium and making said cells to express aspartase and target gene products, wherein said aspartase can increase the amount of the target gene expression.

13. The method as claimed in claim 12, wherein said promoter in step (a) is an IPTG-induced(isopropyl-β-D-thiogalactopyranoside-induced) promoter which regulates the expression of the aspartase gene.

14. The method as claimed in claim 12, wherein said recombinant vector in step (b) includes constitutive promoter or any other regulating promoter.

15. The method as claimed in claim 13, wherein said promoter is constitutive promoter or any other regulating promoter.

16. The method as claimed in claim 14, wherein said promoter is constitutive promoter or any other regulating promoter.

17. The method as claimed in claim 12, wherein said aspartase coding sequences in step (a) are from E. coli, bacteria, yeast, fungus, insect, plant, animal or human.

18. The method as claimed in claim 12, wherein said aspartase coding sequences in step (a) are from E. coli.

19. The method as claimed in claim 12, wherein said target gene in step (d) encodes recombinant protein or polypeptide.

20. The method as claimed in claim 19, wherein said recombinant protein is homogeneous protein or heterogeneous protein.

21. The method as claimed in claim 20, wherein said heterogeneous protein is Aequorea green fluorescent protein.

22. The method as claimed in claim 20, wherein said homogeneous protein is β-galactosidase.

23. The method as claimed in claim 12, wherein the host cells in step (c) are from E. coli, bacteria, yeast, fungus, plant cells, insect cells or mammal cells.

24. The method as claimed in claim 12, wherein the culture medium in step (d) contains aspartate.

25. The method as claimed in claim 24, wherein said medium is LB.

26. The method as claimed in claim 24, wherein said medium contains LB and glucose.

27. The method as claimed in claim 24, wherein said culture medium contains M9, glucose, and aspartate, wherein said 9contains Na₂HPO₄ (6 g/L), KH₂PO4 (3 g/L), NaCl (0.5 g/L), NH₄Cl (1 g/L), MgSO₄·7H₂O (1 mM), CaCl₂ (0.1 mM).

28. The method as claimed in claim 24, wherein said culture medium contains M9, glucose, yeast extract and aspartate, wherein said M9 contains Na₂HPO₄ (6 g/L), KH₂PO₄ (3 g/L), NaCl (0.5 g/L), NH₄Cl (1 g/L), MgSO₄·7H₂O (1 mM), CaCl2 (0.1 mM).

29. A method to promote cell growth, comprising:
(a) inserting one or more aspartase coding sequence into chromosomes of host cells to produce recombinant host cells; and
(b) culturing said host cells in culture medium and making said cells to express aspartase, then promoting said recombinant host cells growth.

30. The method as claimed in claim 29, wherein said aspartase coding sequences in step (a) are from E. coli, bacteria, yeast, fungus, insect, plant, animal or human.

31. The method as claimed in claim 30, wherein said aspartase coding sequences in step (a) are from E. coli.

32. The method as claimed in claim 29, wherein the host cells in step (b) are from E. coli, bacteria, yeast, fungus, plant cells, insect cells or mammal cells.

33. The method as claimed in claim 29, wherein the culture medium in step (c) contains aspartate.

34. The method as claimed in claim 33, wherein said medium is LB.

35. The method as claimed in claim 33, wherein said medium contains LB and glucose.

36. The method as claimed in claim 33, wherein said culture medium contains M9, glucose, and aspartate, wherein said M9 contains Na₂HPO₄ (6 g/L), KH₂PO₄ (3 g/L), NaCl (0.5 g/L), NH₄Cl (1 g/L), MgSO₄ ·7H₂O (1 mM), CaCl₂ (0.1 mM).

37. The method as claimed in claim 33, wherein said culture medium contains M9, glucose, yeast extract and aspartate, wherein said M9 contains Na₂HPO₄ (6 g/L), KH₂PO₄ (3 g/L), NaCl (0.5 g/L), NH₄Cl (1 g/L), MgSO₄·7H₂O (1 mM), CaCl₂ (0.1 mM).

38. A method to increase the amount of the target gene expression, comprising:
(a) constructing a recombinant vector containing aspartase coding sequence and target gene to be expressed;
(b) transforming said recombinant vector in step (a) into host cells and producing recombinant host cells; and
(c) culturing said host cells in step (b) in culture medium and inducing said cells to express aspartase and target gene products, wherein said aspartase can increase the amount of the target gene expression.

39. The method as claimed in claim 38, wherein said aspartase coding sequences in step (a) are from E. coli, bacteria, yeast, fungus, insect, plant, animal or human.

40. The method as claimed in claim 38, wherein said aspartase coding sequences in step (a) are from E. coli.

41. The method as claimed in claim 38, wherein said target gene in step (a) encodes recombinant protein or polypeptide.

42. The method as claimed in claim 41, wherein said recombinant protein is homogeneous protein or heterogeneous protein.

43. The method as claimed in claim 42, wherein said heterogeneous protein is Aequorea green fluorescent protein.

44. The method as claimed in claim 42, wherein said homogeneous protein is β-galactosidase.

45. The method as claimed in claim 38, wherein the host cells in step (b) are from E. coli, bacteria, yeast, fungus, plant cells, insect cells or mammal cells.

46. The method as claimed in claim 38, wherein the culture medium in step (c) contains aspartate.

47. The method as claimed in claim 38, wherein said medium is LB.

48. The method as claimed in claim 38, wherein said medium contains LB and glucose.

49. The method as claimed in claim 38, wherein said culture medium contains M9, glucose, and aspartate, wherein said M9 contains Na₂HPO₄ (6 g/L), KH₂PO₄ (3 g/L), NaCl (0.5 g/L), NH₄Cl (1 g/L), MgSO₄·7H₂O (1 mM), CaCl₂ (0.1 mM).

50. The method as claimed in claim 38, wherein said culture medium contains M9, glucose, yeast extract and aspartate, wherein said M9 contains Na₂HPO₄ (6 g/L), KH₂PO₄ (3 g/L), NaCl (0.5 g/L), NH₄Cl (1 g/L), MgSO₄·7H₂O (1 mM), CaCl₂ (0.1 mM).

51. A method to increase the amount of the target gene expression, comprising:
(a) inserting one or more aspartase coding sequence into chromosomes of host cells to produce recombinant host cells;
(b) constructing a recombinant vector containing target gene to be expressed;
(c) transforming said recombinant vector in step (b) into recombinant host cells in step (a) and producing recombinant host cells; and
(d) culturing said recombinant host cells of steps (c) in culture medium and inducing said recombinant host cells to express aspartase and target gene products, wherein said aspartase can increase the amount of the target gene expression.

52. The method as claimed in claim 51, wherein said aspartase coding sequences in step (a) are from E. coli, bacteria, yeast, fungus, insect, plant, animal or human.

53. The method as claimed in claim 52, wherein said aspartase coding sequences in step (a) are from E. coli.

54. The method as claimed in claim 51, wherein the host cells in step (a) are from E. coli, bacteria, yeast, fungus, plant cells, insect cells or mammal cells.

55. The method as claimed in claim 51, wherein said target gene in step (b) encodes recombinant protein or polypeptide.

56. The method as claimed in claim 55, wherein said recombinant protein is homogeneous protein or heterogeneous protein.

57. The method as claimed in claim 56, wherein said heterogeneous protein is Aequorea green fluorescent protein.

58. The method as claimed in claim 56, wherein said homogeneous protein is β-galactosidase.

59. The method as claimed in claim 51, wherein the culture medium in step (d) contains aspartate.

60. The method as claimed in claim 59, wherein said medium is LB.

61. The method as claimed in claim 59, wherein said medium contains LB and glucose.

62. The method as claimed in claim 59, wherein said culture medium contains M9, glucose, and aspartate, wherein said M9 contains Na₂HPO₄ (6 g/L), KH₂PO₄ (3 g/L), NaCl (0.5 g/L), NH₄Cl (1 g/L), MgSO₄·7H₂O (1 mM), CaCl₂ (0.1 mM).

63. The method as claimed in claim 59, wherein said culture medium contains M9, glucose, yeast extract and aspartate, wherein said M9 contains Na₂HPO₄ (6 g/L), KH₂PO₄ (3 g/L), NaCl (0.5 g/L), NH₄Cl (1 g/L) , MgSO₄·7H₂O (1 mM), CaCl₂ (0.1 mM)


SEQUENCE LISTING (7 sequences)

SEQ ID NO: 1 - DNA, 28 bp, Artificial
  primer
cacaggatccacaacattcgtatcgaag

SEQ ID NO: 2 - DNA, 33 bp, Artificial
  primer
gctaagcttactgttcgctttcatcattatagc

SEQ ID NO: 3 - DNA, 28 bp, Artificial
  primer
taactcgcgataattgcgttgcgctcac

SEQ ID NO: 4 - DNA, 29 bp, Artificial
  primer
cgcccatggtatatctccttcttacaagc

SEQ ID NO: 5 - DNA, 28 bp, Artificial
  primer
acagccatggccatgattacggattcac

SEQ ID NO: 6 - DNA, 28 bp, Artificial
  primer
cggaagcttttatttttgacaccagacc

SEQ ID NO: 7 - DNA, 2040 bp, Escherichia coli
tgatcagcgaaacacttttaatcatctccgccgctgggttttcacccgccgccattttttgctgcatcagcacgaaattcttaaagccctggttacgtaccagtgacataccgataactgacgtgaatataaccagcacgagggtcagcaatacccccaatacatgggcaacctgaataaagattgaaatctcaatatagacataaaggaaaatggcaataaaaggtaaccagcgcaaaggtttctcctgtaatagcagccggttaaccccggctacctgaatgggttgcgaatcgcgtttagcttatattgtggtcattagcaaaatttcaagatgtttgcgcaactatttttggtagtaatcccaaagcggtgatctatttcacaaattaataattaaggggtaaaaaccgacacttaaagtgatccagattacggtagaaatcctcaagcagcatatgatctcgggtattcggtcgatgcaggggataatcgtcggtcgaaaaacattcgaaaccacatatattctgtgtgtttaaagcaaatcattggcagcttgaaaaagaaggttcacatgtcaaacaacattcgtatcgaagaagatctgttgggtaccagggaagttccagctgatgcctactatggtgttcacactctgagagcgattgtaaacttctatatcagcaacaacaaaatcagtgatattcctgaatttgttcgcggtatggtaatggttaaaaaagccgcagctatggcaaacaaagagctgcaaaccattcctaaaagtgtagcgaatgccatcattgccgcatgtgatgaagtcctgaacaacggaaaatgcatggatcagttcccggtagacgtctaccagggcggcgcaggtacttccgtaaacatgaacaccaacgaagtgctggccaatatcggtctggaactgatgggtcaccaaaaaggtgaatatcagtacctgaacccgaacgaccatgttaacaaatgtcagtccactaacgacgcctacccgaccggtttccgtatcgcagtttactcttccctgattaagctggtagatgcgattaaccaactgcgtgaaggctttgaacgtaaagctgtcgaattccaggacatcctgaaaatgggtcgtacccagctgcaggacgcagtaccgatgaccctcggtcaggaattccgcgctttcagcatcctgctgaaagaagaagtgaaaaacatccaacgtaccgctgaactgctgctggaagttaaccttggtgcaacagcaatcggtactggtctgaacacgccgaaagagtactctccgctggcagtgaaaaaactggctgaagttactggcttcccatgcgtaccggctgaagacctgatcgaagcgacctctgactgcggcgcttatgttatggttcacggcgcgctgaaacgcctggctgtgaagatgtccaaaatctgtaacgacctgcgcttgctctcttcaggcccacgtgccggcctgaacgagatcaacctgccggaactgcaggcgggctcttccatcatgccagctaaagtaaacccggttgttccggaagtggttaaccaggtatgcttcaaagtcatcggtaacgacaccactgttaccatggcagcagaagcaggtcagctgcagttgaacgttatggagccggtcattggccaggccatgttcgaatccgttcacattctgaccaacgcttgctacaacctgctggaaaaatgcattaacggcatcactgctaacaaagaagtgtgcgaaggttacgtttacaactctatcggtatcgttacttacctgaacccgttcatcggtcaccacaacggtgacatcgtgggtaaaatctgtgccgaaaccggtaagagtgtacgtgaagtcgttctggaacgcggtctgttgactgaagcggaacttgacgatattttctccgtacagaatctgatgcacccggcttacaaagcaaaacgctatactgatgaaagcgaacagtaatcgtacagggtagtacaaataaaaaaggc